(19) United States
(12) Patent Application Publication
(10) Pub. No.: US 2017/0355942 A1
(43) Pub. Date: Dec. 14, 2017

(54) CYCLIC PHOTOBIOREACTOR AND METHOD FOR BIOFILM CONTROL

(71) Applicant: University of Kentucky Research Foundation, Lexington, KY (US)

(72) Inventors: Michael Hayes Wilson, Lexington, KY (US); John Groppo, Lexington, KY (US); Thomas Grubbs, Lexington, KY (US); Charles Cecil, Lexington, KY (US); Mark Crocker, Lexington, KY (US)

(21) Appl. No.: 15/621,776

(22) Filed: Jun. 13, 2017

Related U.S. Application Data
(60) Provisional application No. 62/349,404, filed on Jun. 13, 2016.

(51) Int. Cl. C12M 1/00; C12M 1/12

(52) U.S. Cl. CPC C12M 21/02; C12M 23/06; C12M 23/40; C12M 37/00; C12M 29/00

(57) ABSTRACT

The invention herein describes a novel photobioreactor (PBR) system for culturing algae. The PBR system utilizes vertical transparent tubes that can be cyclically filled with an algae culture. The vertical tubes further feature pipe pigs deployed therein to disrupt formation of biofilm on the inner walls during the cyclic filling and emptying of the cultures.

### RELATED APPLICATIONS

This application claims priority to U.S. Provisional Patent Application 62/349,404, filed Jun. 13, 2016, all of which is hereby incorporated by reference in its entirety.

### FIELD OF THE INVENTION

The present invention relates to a photobioreactor system for closed-loop production of algae that minimizes biofilm accumulation and energy consumption while providing homogenous harvested cultures.

### BACKGROUND

Algae cultures are currently maintained at commercial scale for a variety of reasons, such as the production of nutrient supplements and biofuel feedstocks, while emerging uses such as bioplastic production are currently being developed. To maximize productivity while ensuring quality control of the algae culture being grown, closed systems such as photobioreactors (PBRs) offer specific advantages over conventional open pond technologies. Open ponds, for example, provide no means of preventing invasive species from being introduced, and hence poly-cultures coexist limiting quality control. Open ponds also require significant water make-up due to evaporative losses, thus making control of soluble nutrient balances complicated. Additionally, since algae require sunlight to grow, dense algae cultures limit sunlight penetration in open ponds to a depth of approximately 5 inches; thus, achieving high productivity requires open ponds to be shallow with a large areal footprint. The invention described herein provides a closed system that allows for production of algae that minimizes biofilm accumulation and energy consumption while providing homogenous harvested cultures.

### SUMMARY OF THE INVENTION

The present invention provides for a photobioreactor (PBR) system of two parallel manifold reactor tube banks that are connected at the top and bottom with pipe pigs deployed within each tube. The arrangement of the tubes allows for both banks to receive solar radiation and minimize shading, thereby encouraging maximal culture growth.

PBRs provide a contained growth environment for algae cultures, thus minimizing contamination from invasive species while eliminating evaporative losses. The PBR system of the present invention comprises clear vertical tubes (to maximize sunlight exposure) arranged in offset parallel rows (to minimize shading). The vertical tubes are filled with the algae culture by means of a pump, however the pump is not operated continuously, rather, only long enough to fill the vertical tubes. In this manner, pumping energy requirements are significantly reduced over conventional PBR systems. Another advantage is that since the culture volume resides in the vertical tubes, multiple parallel rows can be filled using a much smaller feed tank with a single small pump using a system of automated control valves.

To ensure culture homogeneity while maximizing culture exposure to sunlight, each parallel row is periodically (i.e. every 2-6 hours) recycled back to the feed tank where it is thoroughly mixed before being reintroduced back into the vertical tubes. This cyclic filling and draining operation provides the opportunity to remedy a serious problematic limitation of PBRs, namely controlling biofilm formation.

In any growth system, algae cultures have a natural tendency to attach to stationary surfaces, thus forming biofilms. In natural systems such as shallow rivers and lakes, biofilms form on rocks, tree limbs and other surfaces. Biofilms frequently accumulate and build on surfaces, particularly in stagnant or low velocity flow regimes. In PBRs, biofilms form on the tube walls which can be problematic since biofilm formation limits sunlight penetration. Thus, limiting biofilm formation is essential for maintaining growth. If biofilms are allowed to remain on stationary surfaces, the organism will firmly attach itself by secretion. For this reason, it is essential to control biofilm formation by removing organisms accumulated on stationary surfaces before secretion occurs. In the subject invention, biofilm formation is controlled by the use of buoyant pipe pigs.

Each PBR tube contains a pipe pig of two pliable rubber discs attached to each end of a cylinder of buoyant closed-cell foam. As the PBR fills, the pipe pig floats to the top of the tube and accumulated algae cells are mechanically scraped off the tube walls by the rubber discs. As the PBR drains, the pipe pig falls, and the tube walls are scraped again. The cycle is repeated each time the PBR is filled and drained, thus providing an effective means of controlling biofilm formation. This approach has been shown to be effective through pilot demonstration.

In summary, the subject invention provides a means of operating a vertical PBR constructed of clear tubes which offers the specific advantages of:

Low cost, recyclable construction materials such as thin-walled clear PET (polyethylene terephthalate) tubes mounted on rigid PVC (polyvinyl chloride) pipe.

Minimizing pumping energy requirements by cyclicpumping rather than continuous pumping.

Minimizing feed tank size requirements by enabling multiple banks of PBR tubes to be filled with a relatively small feed tank.

Minimizing the number of pumps required for large scale operation by utilizing automated control valves that allow a single pump to drain and fill multiple PBRs.

Unique staggered tube arrangement that maximizes the number of PBR tubes perunit area while minimizing shading.

Providing homogeneous cultures by periodically mixing the volume of each PBR.

Providing a means of biofilm mitigation by the use of buoyant pipe pigs.

### BRIEF DESCRIPTION OF THE DRAWINGS

FIG. 1 shows an overhead view of parallel manifolds showing alignment of tubes to minimize shading.

FIG. 2 shows a computer generated image showing PBR tube arrangement along with a photograph of an installed pilot-scale demonstration.

FIG. 3 shows a process flow diagram of the photobioreactor system.

FIG. 4 shows the assembly of the cyclic flow PBR.

FIG. 5 shows a cross-sectional view of the top portion of the tube assembly.

FIG. 6 shows a cross-sectional view of the bottom portion of the tube assembly.

FIG. 7 shows an image of an assembled PBR with 3.5 inch diameter tubing.

FIG. 8 shows an exploded view of a flanged joint between PBR sections.

FIG. 9 shows an aerial view of a 10,000 litre cyclic-flow PBR reactor.

FIG. 10 shows a graph of measured pH of a culture against the set point.

FIG. 11 shows an image of pipe pigs deployed in tubes of the PBR reactor assembly.

FIG. 12 shows an exploded view of a pipe pig assembly.

FIG. 13 shows a cross-sectional view of the top section of the tube assembly with a pipe pig deployed therein.

FIG. 14 shows a cross-sectional view of the bottom section of the tube assembly with a pipe pig deployed therein.

FIG. 15 shows a comparison of productivity and PAR for a cyclic PBR and a serpentine PBR.

### DETAILED DESCRIPTION

The present invention provides a PBR system of an assembly of two banks of vertically aligned tubes in connection at the top and bottom thereof. The tubes are aligned in a manner so that the two banks of tubes are alternately spaced, such that between the neighboring tubes of one bank, sufficient space exists that a tube from the second bank is partially or entirely exposed. The tubes of both banks are of a nature that allows the passage of light, such as a fully or partially transparent and/or translucent material. The tubes may be of a clear material, such as a transparent polymer.

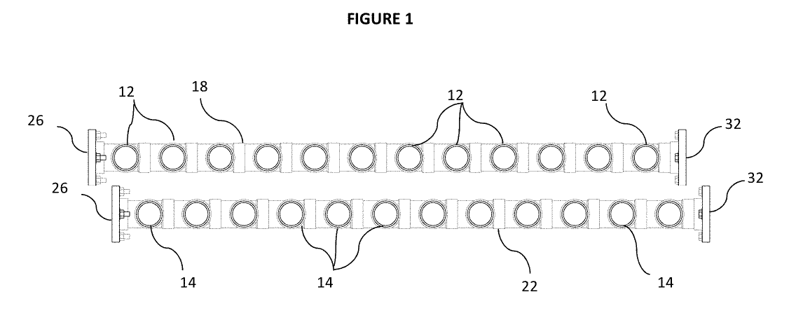

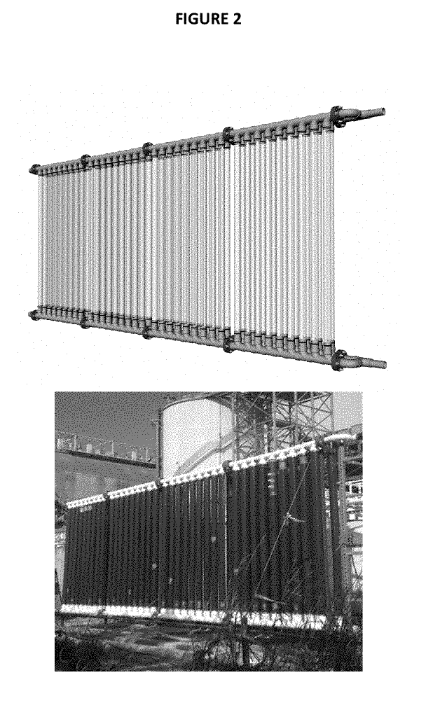

The photobioreactor (PBR) comprises a system of vertical tubes, linked in a closed-loop system. The PBR may be constructed of a clear material, such as PET (polyethylene terephthalate). To increase individual tube access to solar radiation and minimize shading, reactor tubes are arranged in two parallel manifold lines, offset so that each tube of one bank is centered between the empty spaces of the other bank (see e.g. FIG. 1). FIG. 2 depicts a computer generated image of the PBR tube arrangement along with a photograph of an installed pilot-scale demonstration.

The PBR system operates in a ‘cyclic flow’ manner and is designed to operate very differently compared to previous PBR designs. Continuously circulating algae cultures through the phototube array has some unintended and negative consequences. Fully developed flow in a pipe results in a ‘no slip’ condition at the pipe wall. This provides convenient conditions for algae cells to accumulate on the wall of the tube. Given time, these cells will colonize and form a biofilm. The reactor of this invention does not flow continuously and the tubes instead fill and drain in a cyclic manner multiple times per day. As a result, flow never is fully developed and biofilm formation is minimized.

Biofilm mitigation is controlled in this reactor in multiple ways:
a. not flowing the reactor continuously (which also results in energy savings),
b. the introduction of gas bubbles to the reactor body to create multi-dimensional fluid mixing,
c. cycling the flow in order to disrupt the biofilm formation process, and
d. through the use of a buoyant pipe pig to clean the reactor walls as flow is cycled.

Energy savings within the PBR system of the invention are realized by not sparging gas or running a pump continuously, but by duty cycling their operations based on the needs of the algae culture for mixing, ensuring suspension of the culture, and providing adequate CO₂.

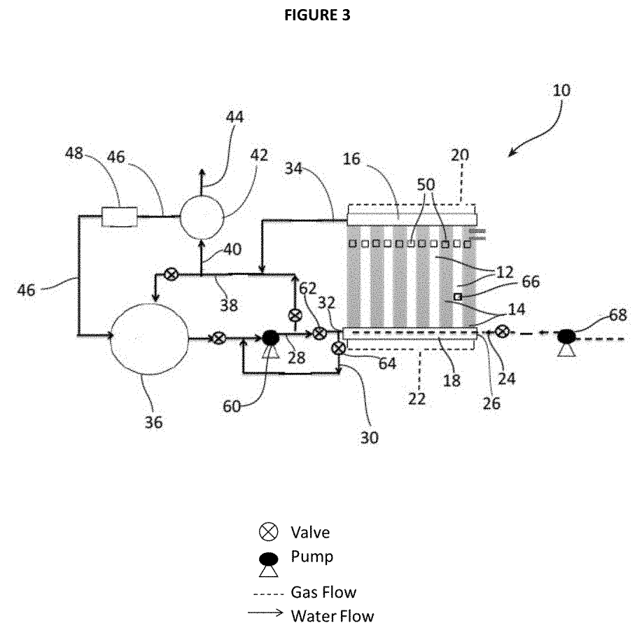

As illustrated in FIGS. 1 and 3, the photobioreactor (PBR) assembly 10 includes a first bank of a plurality of vertical transparent cylindrical tubes 12 (i.e. a phototube array). A second bank of a plurality of vertical transparent tube (not shown) may also be provided wherein the banks are offset such that the center of each tube 12 of the first bank is aligned with space between tubes 14 of the second bank.

An upper horizontal manifold 16 and a lower horizontal manifold 18 are connected to the ends of the first bank of tubes 12.

The lower horizontal manifold 18 is connected to a gas inlet source 24 at one end 26 and a tube fill line 28 and a tube drain line 30 at the other end 32. The upper horizontal manifold 16 is connected to an overflow and/or gas exchange line 34. The overflow line 34 enables gas, originally in the empty tubes 12, 14 to be transferred back to the tank 36 during full cycles and for the same volume of gas to be transferred back to the tubes during draining cycles to prevent suction from damaging the semi-rigid PET tubes 12, 14.

A main process tank 36 is connected to the tube fill line 28, the tube drain line 30 and a process return line 38. The process return line 38 is connected to the tube fill line 28 and a harvest port 40. The main process tank 36 is sized to be equal or greater to the volume of the tubes 14, 16.

The harvest port 40 is connected to a harvest tank 42 having an outlet 44 for biomass removal and a supernate return line 46 that is connected to the main process tank 36. The supernate return line 46 returns clarified water (containing any unused nutrients) to the process tank 36. A UV sterilizer 48, to manage system contamination, lies in the supernate return line 46 between the harvest tank 42 and the main process tank 36.

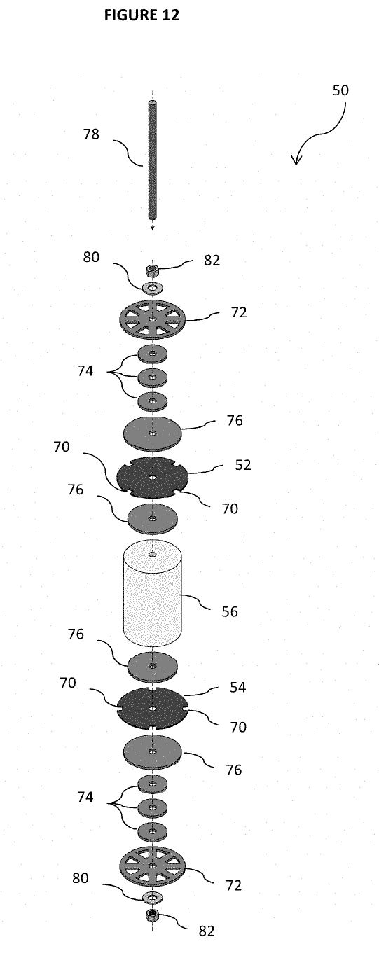

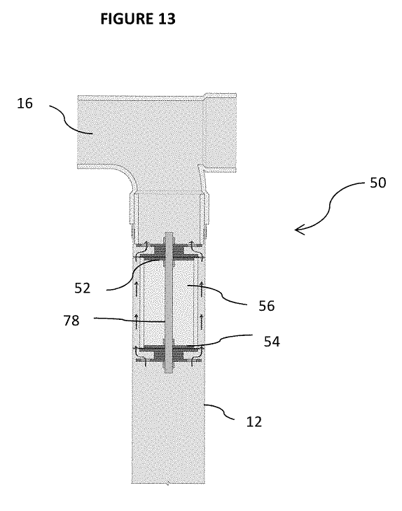

A pipe pig 50 is deployed in each tube 12, 14. Each pipe pig 50 includes an upper circular disc/gasket 52, a lower circular disc/gasket 54 and a buoyant body 56. See FIGS. 12 and 13. The pipe pig 50 functions to prevent the formation of biofilm on the inside of the tubes 12, 14 in a manner described in greater detail below. The discs/gaskets 52, 54 of the pipe pig 50 are notched to allow a liquid culture to pass between the pipe pig and the inner wall of the tube 12, 14 in which the pipe pig is received. Those notches 58 may be placed at 45 degrees with respect to each other.

A pump 60 is provided between the main process tank 36 and the lower horizontal manifold 18. At least one valve 62 is provided along the tube fill line 28. At least one valve 64 is provided along the tube drain line 30. The pump 60 and various valves 62, 64 are used to move algae slurry from the process tank 36 to the tubes 12, 14 (aka phototube arrays) and periodically back to the tank 36 via the return/recycle line 38 for mixing and to tank 42 for harvesting.

CO₂ gas is periodically added directly to the tubes 12, 14 by the pump 68 in order to mix the culture and adjust the pH. Further, a probe 66 is deployed within at least one vertical tube 12, 14 in order to monitor pH, carbon dioxide and/or oxygen levels of the liquid culture in the tubes. The feed and drain valves 62, 64 are repeated for each additional tube array to accommodate a series of parallel reactors to operate separately.

Water, further comprising a seed culture, and further optional nutrients can be added to the main process tank 36 and mixed via a centrifugal pump (not shown) prior to the resulting algae slurry being sent to fill the phototube array through the lower outlet(s). This process is repeated until all of the phototube arrays are filled. The vertical phototubes 12, 14 create a quiescent water column which provides the algae cultures therein with access to photoactive radiation, which is an enhanced environment for photosynthesis. The cultures within the phototubes 12, 14 may then be periodically sparged with flue gas (or other CO₂-containing gas) in order to provide the culture with CO₂, mix the culture, control pH, and provide multidimensional fluid flow to control biofilm. Further, multiple times per day, the entire volume in the phototube array may be drained to disrupt biofilm formation, enable mixing of the culture to maintain homogeneity of the system, and to actuate the pipe pigs deployed in each tube to scrape biofilm from the phototubes. The separate phototube arrays may essentially share a main process tank with the periodic draining and mixing set on a predetermined duty cycle.

### Pipe Pig Assembly and Use

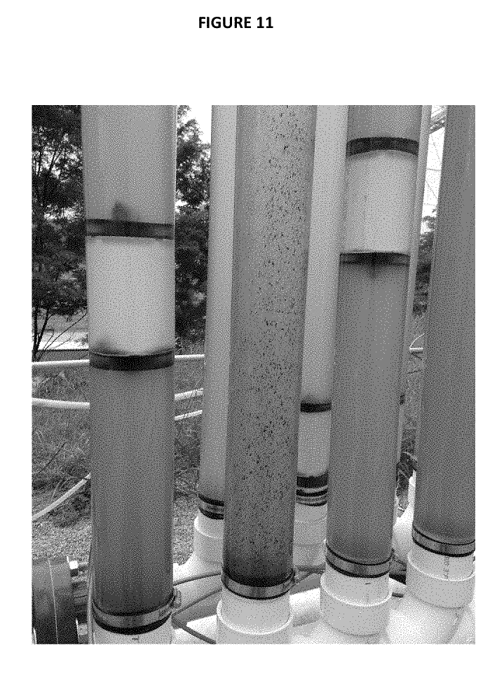

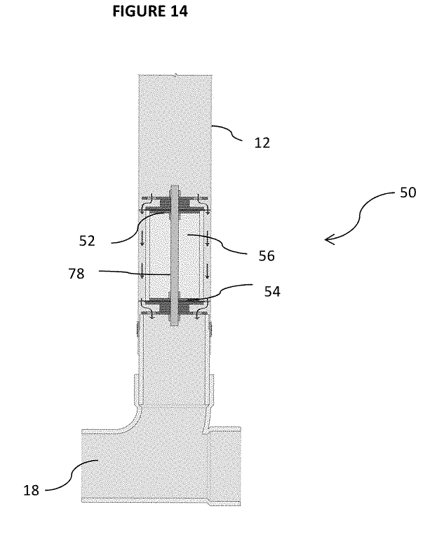

The PBR system provides for one or more pipe pigs 50 deployed within each vertical tube 12, 14, such as at the top and/bottom of each tube. The purpose of the pipe pigs 50 is to prevent the formation of biofilm on the inside of the PET tubes 12, 14, as shown in FIG. 11. The pipe pigs 50 may comprise an upper and/or lower circular disc(s)/gasket(s) 52, 54, the circumference of which is roughly equal to or lightly less than the inner circumference of the tube within which it is deployed. In order to accomplish the task of preventing biofilm formation, each pipe pig 50 must rise to the top of its corresponding tube 12, 14 when the row of tubes is filled with water, and must return to the bottom of the tube when the row is emptied. The body 56 of the pipe pig 50 may comprise, for example, a cylindrical section of foam or other body less dense than the resulting liquid to which it will be exposed (FIG. 12), being large enough that the buoyancy of the pig is sufficient for it to rise and fall with the water level inside the tube. As the pipe pig 50 rises (and falls), the edges of its discs, e.g. two rubber gaskets 52, 54, remove by frictional contact any algae that have accumulated on the inner walls of the phototubes 12, 14. However, while these gaskets 52, 54 should remain in contact with the edge of the tubes 12, 14, they must not restrict the flow of water around the pig 50. To prevent such a restriction, each disc/gasket may be notched several times 70 to allow for sufficient water flow during operation. One disc/gasket is then rotated 45° from the other to ensure a seamless cleaning surface. Each pipe pig 50 may also feature a mechanical stop 72 at each end (FIG. 12) to prevent the pig 50 from leaving the PET tube 12, 14 while still allowing water to flow in and out of the top and bottom tube manifolds 16, 18, 20, 22, as shown in FIG. 13 and FIG. 14, respectively. Finally, the entire pipe pig assembly 58 may be held together by a threaded rod 78 and matching washers 80 and nuts 82. The pipe pig 50 may also include spacers 74 and gasket washers 76.

### Operating Principle

Water, further comprising a seed culture and nutrients can be added to the main process tank 36 and mixed via a centrifugal pump (not shown) prior to the algae slurry being sent to fill a phototube array 12, 14. This process can be repeated until all of the phototube arrays 12, 14 are filled for normal operation. The vertical phototubes 12, 14 may create a quiescent water column providing the algae with access to photoactive radiation, an enhanced environment for photosynthesis. The culture can be periodically sparged with CO₂-containing gas in order to provide CO₂, mix the culture, control pH, and provide multidimensional fluid flow to control biofilm. Multiple times per day, the entire volume in the phototube array 12, 14 is drained to disrupt biofilm formation, enable mixing of the culture to maintain homogeneity of the system, and to actuate pipe pigs 50 in each tube. The separate phototube arrays 12, 14 essentially share a main process tank 36 with the periodic draining and mixing set on a predetermined duty cycle. Energy savings are realized by not sparging gas or running a pump continuously, but by duty cycling their operations based on the needs of the algae culture for mixing, ensuring suspension of the culture, providing adequate CO₂, and actuation of the pipe pigs to control biofilm mitigation.

CO₂ is typically fed to the PBR based on the system's current pH. As CO₂ is fed to the reactor, it forms carbonic acid, which in turn lowers the pH of the algae culture. As photosynthesis occurs, the dissolved CO₂ is consumed and the pH of the system rises. This approach maintains the system pH within an optimum pH range for algal growth while providing enough CO₂ to sustain growth. This is particularly important if the CO₂ source is coal combustion flue gas which may contain other acidic components such as SOₓ and NOₓ. The dissolution of SOx in particular, which forms H₂SO₃/H₂SO₄, can result in over-acidification of the culture medium, thereby inhibiting growth. For this reason, it is important that SOx is not added to the cultivation system faster than its dissolution products can be utilized by the algae.

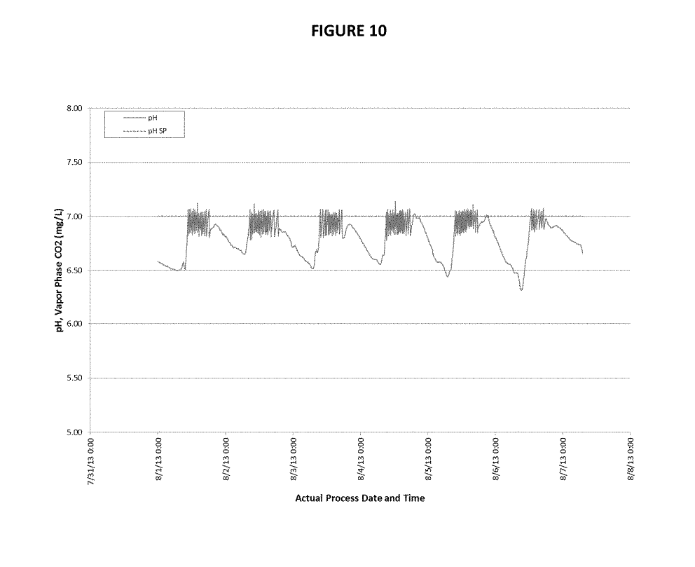

FIG. 10 illustrates the pH control method used to regulate CO₂ flow to the reactor during six days of algal cultivation in a 650 L PBR. The horizontal line indicates the pH set point of the reactor, while the trace shows the measured pH of the system. This graph also captures the occurrence of respiration, which produces CO₂, thereby lowering the pH during the night hours.

Alternatively, CO₂-containing gas can be sparged into the PBR according to a fixed duty cycle, e.g., for a fixed number of seconds during each minute. While this does not permit accurate control of the culture pH, it facilitates regular mixing of the culture in each PBR tube regardless of the rate of culture growth.

In addition to pH, a variety of process parameters are constantly monitored during operation to track the performance of the photobioreactor and the health of the algae culture. These measurements (including pH) are conducted using a series of probes (66) which monitor the algae culture. Temperature, both environmental and within the process, is measured to correlate the performance of the system with environmental effects. The amount of CO₂ in the gas phase may be tracked using gas phase CO₂ sensors and can be correlated with dissolved CO₂ sensors to ensure the system has enough CO₂ to drive algae growth. If desired, photosynthetically active radiation (PAR) can be quantified using a quantum sensor that measures the flux of photons in the photosynthetic spectrum. Dissolved O₂ sensors are used to track the product of the photosynthetic reaction and ensure that there is not an excess of O₂ dissolved in the system. The O₂ generated is near-equivalent on a stoichiometric basis to the CO₂ consumed. Hence, by monitoring the dissolved oxygen, the performance and health of the algae culture can be determined.

### EXAMPLES

### Materials

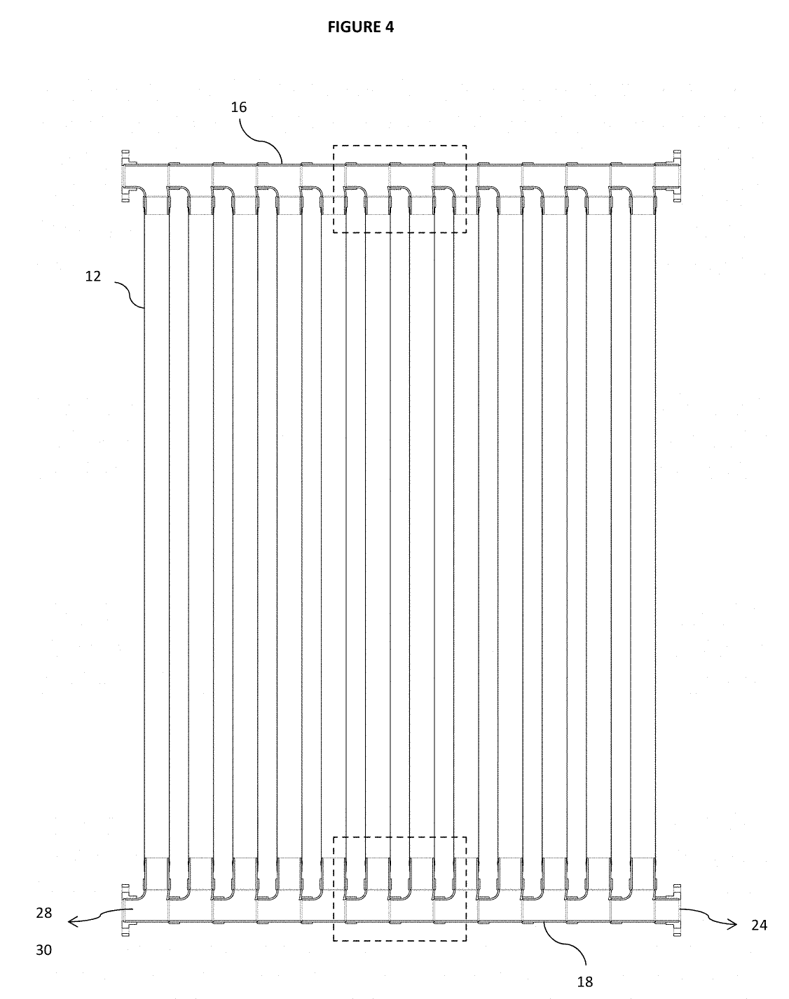

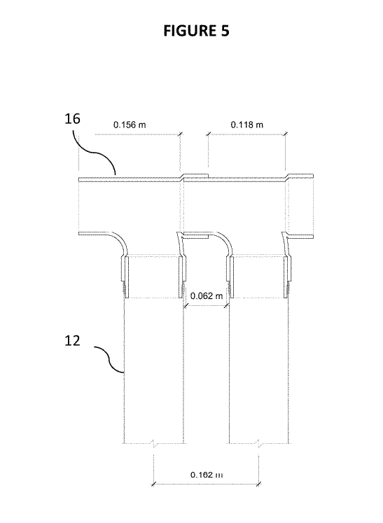

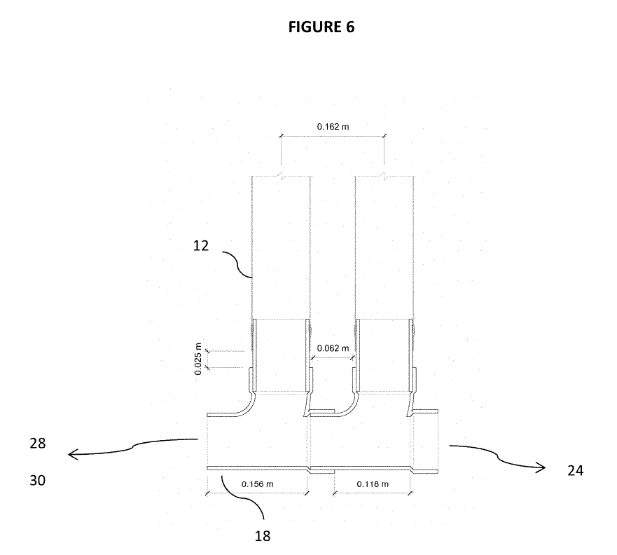

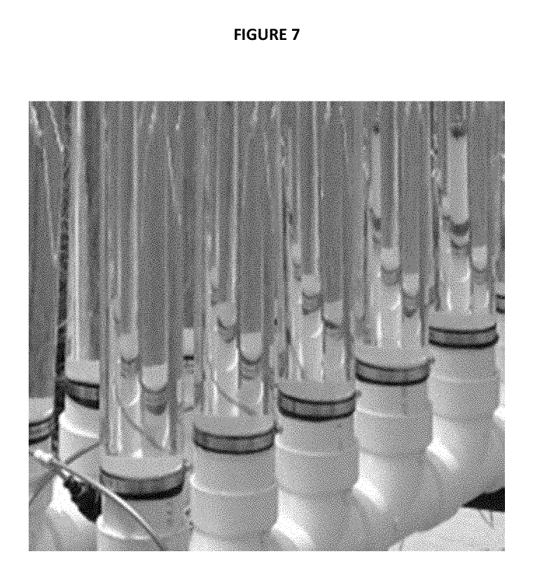

PET tubes (8.9 cm diameter×244 cm high) are vertically aligned and connected by 7.5 cm diameter schedule 40 PVC (polyvinyl chloride) pipe. The thin wall PET tubes, more commonly used as packaging materials, are matched with schedule 40 3″ PVC fittings to create a photoactive reactor body (see e.g. FIG. 4). In order to ensure a watertight seal, appropriately sized rubber bands are used to create gaskets between the tube and the fitting. A worm drive band clamp is used to reinforce the seal. The vertical tubes are supported from the bottom, rather than hanging from the top; supporting the reactor bodies from the bottom forces the weight of the water to act as a compressive force on the reactor, thereby holding it together, rather than in tension which would elongate the tubes causing premature failure. This requires the seal to only withstand the hydrostatic pressure of the water. FIG. 5 shows a schematic of the top part of the tube subassembly, while a schematic and a photograph of the bottom part of the subassembly are provided in FIG. 6 and FIG. 7, respectively.

### Productivity Comparison

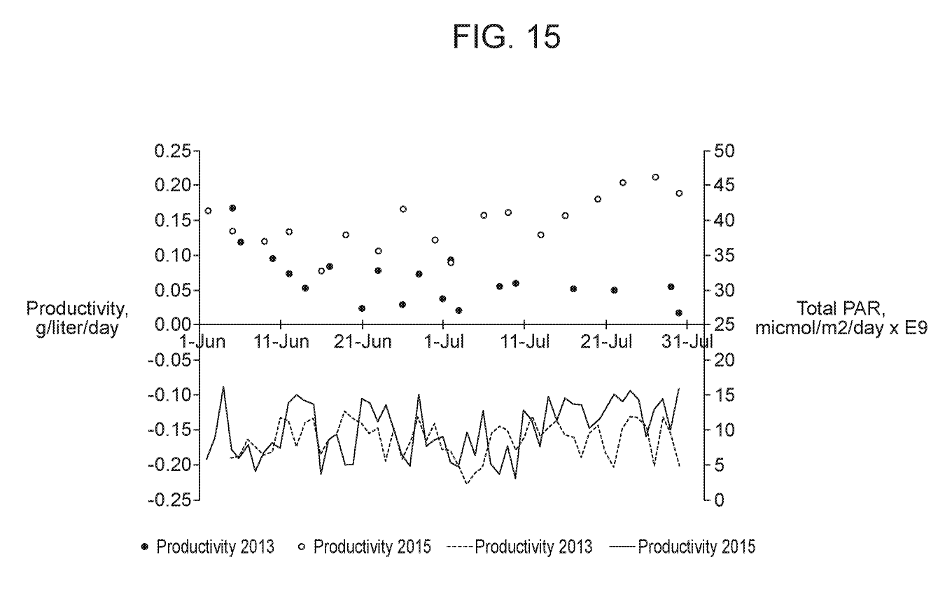

Two different types of PBRs were operated using flue gas from a coal-fired utility as a source of CO₂. The cyclic flow PBR was demonstrated in 2015 while during 2013, a continuous flow PBR was used where the algae culture was continuously pumped through a series of tubes linked in a continuous serpentine flow path. Comparative productivity results along with total PAR for the months of June and July are shown in FIG. 15. For the cyclic flow PBR (2015), productivity was 0.08 to 0.17 g/liter/day throughout June and increased to 0.13 to 0.22 g/liter/day during July. For the serpentine PBR (2013), initial productivity was comparable but decreased to 0.2 to 0.9 g/liter/day later in the month and remained below 0.6 g/liter/day until mid-July, followed by no productivity primarily due to severe biofilm formation.

### Expansion of Reactor Design to Larger Scales

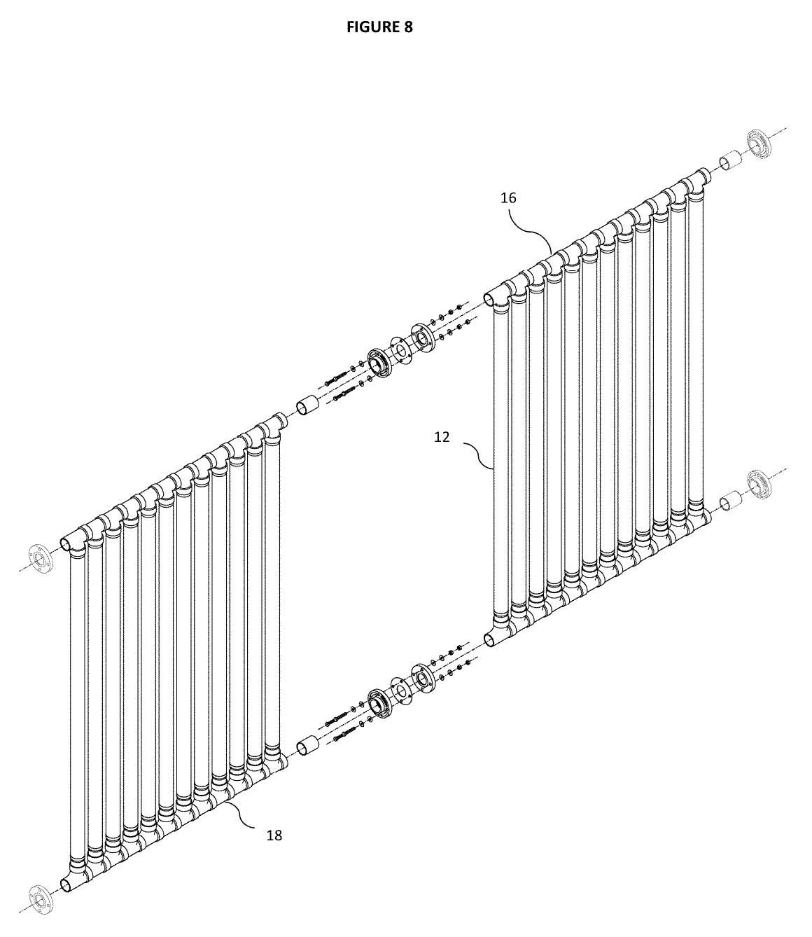

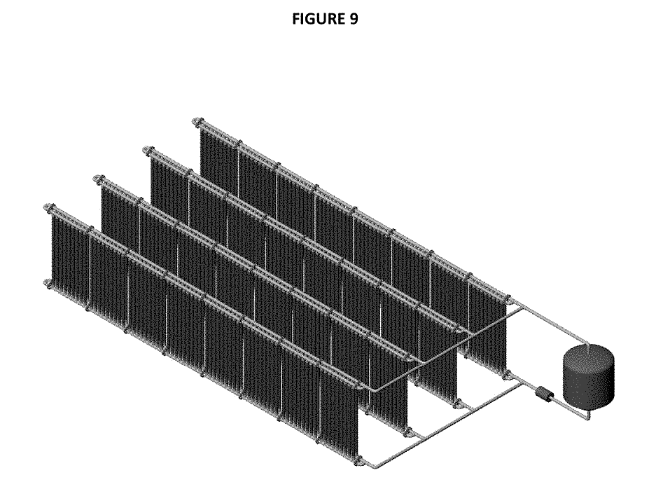

The basic PBR system can be expanded upon by extending the rows of tubes, thereby increasing the photoactive volume of the culture. This is achieved by connecting pre-assembled tube banks (e.g., 12 tubes) using a series of flanged connections, as shown in FIG. 8. If extending the length of the row of tubes is considered to be expansion along the Y axis, expansion along the X axis can be achieved by operating multiple tube rows parallel to one another at a fixed interval (based upon local solar angle and availability of space in order to achieve maximum solar access), as demonstrated in Fig.9.

### Energy Consumption Comparison

A comparison of pumping requirements for the serpentine and cyclic flow PBRs is shown in Table 1. While the serpentine PBR requires continuous flow throughout the day, the cyclic PBR requires pumping only once every 4 hours and each cycle lasts only 14 minutes, thus the total time that pumping is required in only 84 minutes per day. Thus, the cyclic PBR requires only 5.8% of the pumping requirements of a continuous flow PBR, representing a 94.2% reduction in energy costs associated with pumping.

TABLE 1
Comparison of Pumping Requirements for Cyclic and Serpentine PBRs

| | Cyclic PBR | Serpentine PBR |
|---|---|---|
| Pumping Requirements | Once/4 hrs | Continuous |
| Pumping Cycle Duration | 14 minutes | Continuous |
| Pumping Requirements, minutes/day | 84 | 1440 |
| Pumping Requirements, % of day | 5.8 | 100 |

The foregoing has been presented for purposes of illustration and description. It is not intended to be exhaustive or to limit the embodiments to the precise form disclosed. Obvious modifications and variations are possible in light of the above teachings. All such modifications and variations are within the scope of the appended claims when interpreted in accordance with the breadth to which they are fairly, legally and equitably entitled. All documents referenced herein including patents, patent applications and journal articles are hereby incorporated by reference in their entirety.

1. A photobioreactor (PBR) assembly comprised of
a first bank of a plurality of vertical transparent cylindrical tubes;
a pipe pig deployed within each tube;
an upper horizontal manifold and a lower horizontal manifold on said bank of vertical tubes in connection with each end of the vertical tubes, wherein the lower horizontal manifold is connected to a gas inlet source at a first end and a tube fill line and a tube drain line at a second end and wherein one upper horizontal manifold is connected to an overflow and/or gas exchange line; and,
a main process tank connected to the tube fill line and the tube drain line and a process return line, wherein the process return line is connected to the tube fill line and a harvest port.

2. The PBR assembly of claim 1, wherein the harvest port is connected to a harvest tank with an outlet for biomass removal and a supernate return line that is connected to the main process tank.

3. The PBR assembly of claim 2, wherein a UV sterilizer lies in the supernate return line between the harvest tank and the main process tank.

4. The PBR assembly of claim 1, wherein the pipe pig comprises at least one disc at each end of the pipe pig with a diameter of about the inner diameter of the vertical tube in which it is deployed affixed to a buoyant material with a density such that the pipe pig will float in water.

5. The PBR assembly of claim 4, wherein the disc of the pipe pig is notched to allow a liquid culture to pass between the pipe pig and an inner wall of the tube.

6. The PBR assembly of claim 5, wherein the notches in the discs at each end of the pipe pig are placed at 45 degrees with respect each other.

7. The PBR assembly of claim 4, wherein a further notched disc is placed at each end of the pipe pig to entrap the pipe pig within the vertical tube.

8. The PBR assembly of claim 1, further comprising a pump in between the main process tank and the lower horizontal manifold.

9. The PBR assembly of claim 1, further comprising at least one valve along the tube fill line.

10. The PBR assembly of claim 1, further comprising at least one valve along the tube drain line.

11. The PBR assembly of claim 1, further comprising a probe deployed within at least one vertical tube to monitor pH, carbon dioxide and/or oxygen levels.

12. The PBR assembly of claim 1, further comprising a second bank of a plurality of vertical transparent cylindrical tubes, wherein said second bank of a plurality of vertical transparent tubes are offset such that a center of each tube of said first bank is aligned with space between tubes of said second bank.

13. A method for growing and harvesting algae cultures in the PBR assembly of claim 1, comprising:
moving a liquid culture slurry from the main process tank to fill the plurality of vertical transparent cylindrical tubes via the tube fill line;
closing the tube fill line to maintain the liquid culture slurry in the plurality of vertical transparent cylindrical tubes;
providing a source of solar radiation or artificial illumination to the plurality of vertical transparent cylindrical tubes; and
opening the tube drain line after a period of time to return the liquid culture slurry to the main process tank.

14. The method of claim 13, wherein the period of time is between two and six hours.

15. The method of claim 13, wherein the tube drain line returns the liquid culture to the main process tank via a harvest tank.

16. The method of claim 13, wherein the tube drain line returns the supernate liquid from the harvest tank to the main process tank via a UV sterilizer.

17. The method of claim 13, further comprising adding a seed culture, water and/or nutrients to the main process tank prior to moving the liquid culture to the plurality of vertical transparent cylindrical tubes.

18. The method of claim 13, wherein flow of the liquid culture slurry in and out of the vertical tubes allows the pipe pig to disrupt formation of biofilm therein.

19. The method of claim 13, further comprising pumping carbon dioxide-containing gas through the gas inlet source to adjust the pH of the liquid culture slurry.

20. The method of claim 13, further comprising pumping carbon dioxide-containing gas through the gas inlet source according to a fixed duty cycle.
